(19) United States
(12) Patent Application Publication
(10) Pub. No.: US 2012/0232392 A1
(43) Pub. Date: Sep. 13, 2012

(54) ULTRASOUND DIAGNOSTIC APPARATUS

(75) Inventors: Tsuyoshi TANABE, Kanagawa (JP); Yuji OHSHIMA, Kanagawa (JP)

(73) Assignee: FUJIFILM CORPORATION, Tokyo (JP)

(21) Appl. No.: 13/371,970

(22) Filed: Feb. 13, 2012

(30) Foreign Application Priority Data
Mar. 10, 2011 (JP) 2011-052537
Mar. 25, 2011 (JP) 2011-067381

(51) Int. Cl. A61B 8/14

(52) U.S. Cl. 600/443

(57) ABSTRACT

An ultrasound diagnostic apparatus is provided which includes an ultrasound probe performing ultrasound transmission and reception in different directions and a diagnostic apparatus body combining images different in the direction of transmission and reception to produce an ultrasound image. At least one of ultrasound images to be combined is changed depending on the set region of interest (ROI) to an image of the depth corresponding to the ROI. When an acoustic coupler is attached, the depth of at least one of ultrasound images to be combined is increased. The ultrasound diagnostic apparatus is capable of improving the image quality of the ROI and obtaining efficient high-definition ultrasound images reaching a predetermined depth when the acoustic coupler is attached.

### BACKGROUND OF THE INVENTION

The present invention relates to an ultrasound diagnostic apparatus. The invention more particularly relates to an ultrasound diagnostic apparatus capable of improving the image quality of a region of interest and obtaining a high-definition ultrasound image showing the vicinity of the skin surface of a subject with high efficiency.

Ultrasound diagnostic apparatuses using ultrasound images are put to practical use in the medical field.

In general, an ultrasound diagnostic apparatus includes an ultrasound probe (hereinafter referred to as “probe”) and a diagnostic apparatus body. In the ultrasound diagnostic apparatus, the probe transmits ultrasonic waves toward a subject and receives ultrasonic echoes from the subject. The diagnostic apparatus body electrically processes the reception signals received by and outputted from the probe to produce an ultrasound image.

The probe performs transmission and reception of ultrasonic waves and includes a piezoelectric unit for outputting reception signals (electric signals).

Recently, the probe may also be provided with an integrated circuit board for use in amplifying the reception signals outputted from the piezoelectric unit, performing A/D conversion or other processing, changing the timing of transmission and reception of ultrasonic waves in the piezoelectric unit, wireless communication with the diagnostic apparatus body without using any cord, and reducing noise.

So-called “speckle” (speckle noise/speckle pattern) is known as a factor that may deteriorate the quality of an ultrasound image in the ultrasound diagnostic apparatus. Speckle is white spot noise caused by the mutual interference of scattered waves generated by numerous scattering sources which are present in a subject and have a smaller wavelength than that of an ultrasonic wave.

Spatial compounding as described in JP 2005-58321 A and JP 2003-70786 A is known as a method of reducing such speckle in the ultrasound diagnostic apparatus.

As conceptually shown in FIG. 9, spatial compounding is a technique which involves performing a plurality of types of ultrasound transmission and reception in mutually different directions (at mutually different scanning angles) between a piezoelectric unit 100 and a subject, and combining ultrasound images obtained by the plurality of types of ultrasound transmission and reception to produce a composite ultrasound image.

More specifically, in the example shown in FIG. 9, three types of ultrasound transmission and reception are performed which include the ultrasound transmission and reception as in the normal ultrasound image generation (normal transmission and reception), the ultrasound transmission and reception in a direction inclined by an angle of θ with respect to the direction of the normal transmission and reception, and the ultrasound transmission and reception in a direction inclined by an angle of −θ with respect to the direction of the normal transmission and reception.

An ultrasound image A (solid line) obtained by the normal transmission and reception, an ultrasound image B (broken line) obtained by the transmission and reception in the direction inclined by the angle of θ, and an ultrasound image C (chain line) obtained by the transmission and reception in the direction inclined by the angle of −θ are combined to produce a composite ultrasound image covering the region of the ultrasound image A shown by the solid line.

In the ultrasound diagnostic apparatuses, a so-called near field is more likely to deteriorate the image quality of ultrasound images due to sound speed disturbances and multiple reflection. The near field is a region of the subject near the probe, that is, an extremely shallow region in the direction of ultrasound transmission and reception.

In order to solve this problem, JP 2006-95151 A describes an ultrasound diagnostic apparatus which performs spatial compounding only for the near field to improve the image quality of the near field.

### SUMMARY OF THE INVENTION

According to the ultrasound diagnostic apparatus described in JP 2006-95151 A, ultrasound images with improved near field image quality can be obtained by making use of spatial compounding.

However, in the ultrasound diagnostic apparatus, the region of interest (ROI) to be noted for the importance in diagnosis is not limited to the near field. In other words, regions with different depths may be used as the ROI in the ultrasound diagnostic apparatus.

An object of the present invention is to solve the foregoing prior art problems and to provide an ultrasound diagnostic apparatus capable of improving the image quality of an arbitrary ROI by making use of spatial compounding and of reducing useless reception signal processing and ultrasound scanning (sound rays).

The ultrasound diagnostic apparatus may use an acoustic coupler to focus the ultrasonic waves (ultrasonic beams) near the skin surface of a subject. The acoustic coupler is made of a material having an acoustic impedance close to that of a living body and is attached to the ultrasound transmission and reception surface of a probe.

The attachment of the acoustic coupler enables the ultrasound transmission and reception surface to be kept apart from the skin surface of the subject by a predetermined distance. Therefore, ultrasound images in which the ultrasonic waves are focused near the skin surface of the subject can be obtained by using the acoustic coupler.

Another object of the invention is to provide an ultrasound diagnostic apparatus capable of efficiently obtaining effective ultrasound images showing the vicinity of the skin surface of a subject by making use of spatial compounding even when an acoustic coupler is used.

In order to achieve the first object, a first aspect of the invention provides an ultrasound diagnostic apparatus comprising:
an ultrasound probe configured to transmit ultrasonic waves into a subject and receive ultrasonic echoes generated by reflection of the ultrasonic waves from the subject, the ultrasound probe including a signal processor for processing reception signals based on the ultrasonic echoes; and
a diagnostic apparatus body configured to generate ultrasound images in accordance with the reception signals processed in the signal processor of said ultrasound probe and set a region of interest which is spaced apart from said ultrasound probe,
wherein said ultrasound probe is configured to perform a plurality of types of ultrasound transmission and reception in mutually different directions of ultrasound transmission and reception and said diagnostic apparatus body is configured to combine ultrasound images based on each of the plurality of types of ultrasound transmission and reception, and
wherein, upon production of the composite ultrasound image in said diagnostic apparatus body, said ultrasound probe is configured to control drive of said signal processor so that a depth of at least one of said ultrasound images to be combined is changed according to the region of interest.

In the ultrasound diagnostic apparatus according to the first aspect of the invention, upon the production of the composite ultrasound image in the diagnostic apparatus body, the ultrasound probe preferably performs ultrasound transmission and reception for obtaining a main image as an ultrasound image in a preset predetermined output region by one of the plurality of types of ultrasound transmission and reception.

Upon change of a reception depth of at least one image of the ultrasound images to be combined in accordance with the region of interest, the ultrasound probe preferably does not perform ultrasound scanning in a region of the at least one image having the changed reception depth where the at least one image and the main image do not overlap each other.

Preferably, the ultrasound diagnostic apparatus comprises a temperature sensor for measuring a temperature at a predetermined position inside the ultrasound probe and, upon the production of the composite ultrasound image in the diagnostic apparatus body, the ultrasound probe changes conditions of the ultrasound transmission and reception so as to change an image quality of an ultrasound image to be combined with the main image in accordance with a temperature measurement result obtained with the temperature sensor.

The temperature sensor preferably measures the temperature of the signal processor.

The ultrasound probe preferably transmits and receives ultrasonic waves in identical directions for a last ultrasound image of one composite ultrasound image in temporally consecutive composite ultrasound images and a first ultrasound image of its subsequent composite ultrasound image.

In order to achieve the second object, a second aspect of the invention provides an ultrasound diagnostic apparatus comprising:
an ultrasound probe configured to transmit ultrasonic waves into a subject and receive ultrasonic echoes generated by reflection of the ultrasonic waves from the subject, the ultrasound probe including a signal processor for processing reception signals based on the ultrasonic echoes;
a diagnostic apparatus body configured to generate ultrasound images in accordance with the reception signals processed in the signal processor of said ultrasound probe;

an acoustic coupler detachably attached to said ultrasound probe so as to cover an ultrasound transmission and reception surface of said ultrasound probe; and
a detector provided in at least one of said ultrasound probe and said diagnostic apparatus body to detect that said acoustic coupler is attached to said ultrasound probe,
wherein said ultrasound probe is configured to perform a plurality of types of ultrasound transmission and reception in mutually different directions of ultrasound transmission and reception and said diagnostic apparatus body is configured to combine ultrasound images based on each of the plurality of types of ultrasound transmission and reception, and
wherein, upon production of the composite ultrasound image in said diagnostic apparatus body, said ultrasound probe control drive of said signal processor so that a depth of at least one of said ultrasound images to be combined is changed upon detection of attachment of the acoustic coupler to said ultrasound probe made by said detector.

In the ultrasound diagnostic apparatus according to the second aspect of the invention, upon the detection of the attachment of the acoustic coupler to the ultrasound probe made by the detector, the ultrasound probe preferably increases the depth of at least one of the ultrasound images to be combined.

Upon the production of the composite ultrasound image in the diagnostic apparatus body, the ultrasound probe preferably performs ultrasound transmission and reception for obtaining a main image as an ultrasound image in a preset predetermined output region by one of the plurality of types of ultrasound transmission and reception.

Upon the detection of the attachment of the acoustic coupler to the ultrasound probe made by the detector, the ultrasound probe preferably does not process the reception signals in the signal processor as for a depth region corresponding to the acoustic coupler in at least one of the ultrasound images to be combined.

Upon the detection of the attachment of the acoustic coupler to the ultrasound probe made by the detector, the ultrasound probe preferably does not process the reception signals in the signal processor as for the depth region corresponding to the acoustic coupler in all of the ultrasound images for use in producing the composite ultrasound image.

Upon the detection of the attachment of the acoustic coupler to the ultrasound probe made by the detector, the ultrasound probe preferably does not perform ultrasound scanning of regions of other ultrasound images than the main image where the other ultrasound images and the main image do not overlap each other.

The ultrasound diagnostic apparatus preferably includes a proximity mode for combining the ultrasound images in a predetermined depth region on a subject skin surface side.

Upon the detection of the attachment of the acoustic coupler to the ultrasound probe made by the detector, the ultrasound probe preferably sets the depth of the at least one of the ultrasound images to be combined as a predetermined depth deeper than in the predetermined depth region in the proximity mode.

According to the ultrasound diagnostic apparatus in the first aspect of the invention configured as described above, spatial compounding which involves combining a plurality of images different in the direction of ultrasound transmission and reception is utilized and an arbitrary depth region which is spaced apart from the piezoelectric unit by a predetermined distance or more is treated as the region of interest (ROI), whereby the ROI image quality can be improved. Therefore, the ultrasound diagnostic apparatus of the invention is capable of making a proper diagnosis while showing a region to be noted on an ultrasound image with high definition.

The drive of the signal processor for processing the reception signals outputted from the piezoelectric unit which performs the ultrasound transmission and reception is controlled to generate ultrasound images each having a depth corresponding to an ROI, whereby the processing of useless reception signals which are not involved in the image composition can be reduced.

According to the ultrasound diagnostic apparatus in the second aspect of the invention configured as described above, also in the case of using the acoustic coupler for focusing ultrasonic waves near the skin surface of a subject, effective high-definition ultrasound images can be efficiently obtained by making use of spatial compounding for combining a plurality of images different in the direction of ultrasound transmission and reception.

### BRIEF DESCRIPTION OF THE DRAWINGS

FIG. 1 is a conceptual block diagram showing an ultrasound diagnostic apparatus according to a first aspect of the invention.

FIG. 2 is a conceptual diagram for illustrating spatial compounding that may be performed in the ultrasound diagnostic apparatus shown in FIG. 1.

FIGS. 3A, 3B and 3C are conceptual diagrams for illustrating an example of spatial compounding which is performed in the ultrasound diagnostic apparatus according to the first aspect of the invention.

FIGS. 4A and 4B are conceptual diagrams for illustrating another example of spatial compounding which is performed in the ultrasound diagnostic apparatus according to the first aspect of the invention.

FIG. 5 is a conceptual block diagram showing the ultrasound diagnostic apparatus according to the second aspect of the invention.

FIGS. 6A, 6B, 6C, 6D and 6E are conceptual diagrams for illustrating an example of spatial compounding which is performed in the ultrasound diagnostic apparatus according to the second aspect of the invention.

FIGS. 7A, 7B and 7C are conceptual diagrams for illustrating another example of spatial compounding which is performed in the ultrasound diagnostic apparatus according to the second aspect of the invention.

FIG. 8 is a conceptual diagram for illustrating yet another example of spatial compounding which is performed in the ultrasound diagnostic apparatus according to the second aspect of the invention.

FIG. 9 is a conceptual diagram for illustrating spatial compounding.

### DETAILED DESCRIPTION OF THE INVENTION

Next, the ultrasound diagnostic apparatus of the invention is described in detail by referring to the preferred embodiments shown in the accompanying drawings.

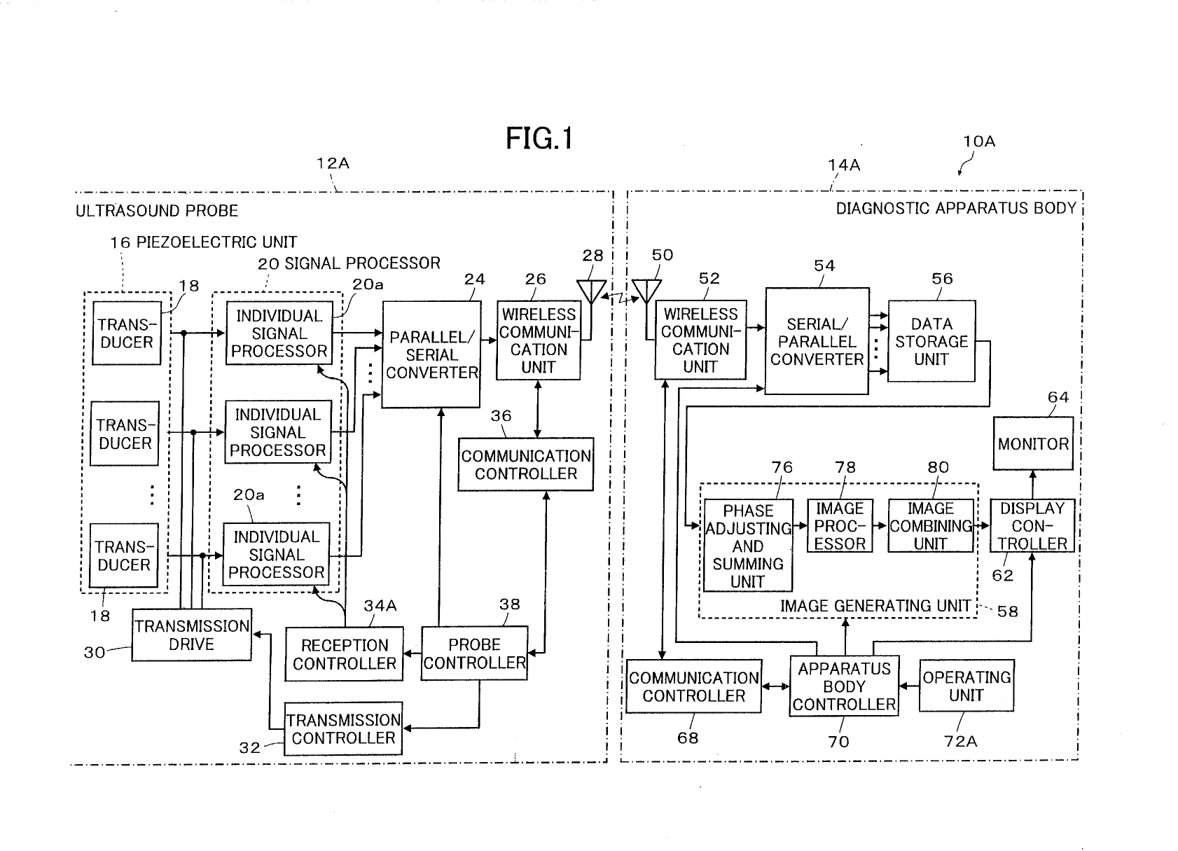

FIG. 1 is a conceptual block diagram showing an embodiment of the ultrasound diagnostic apparatus according to the first aspect of the invention.

An ultrasound diagnostic apparatus 10A shown in FIG. 1 includes an ultrasound probe 12A and a diagnostic apparatus body 14A. The ultrasound probe 12A is connected to the diagnostic apparatus body 14A by wireless communication.

The ultrasound probe 12A (hereinafter referred to as “probe 12A”) transmits ultrasonic waves to a subject, receives ultrasonic echoes generated by reflection of the ultrasound waves on the subject, and outputs reception signals of an ultrasound image in accordance with the received ultrasonic echoes.

In the practice of the invention, various known ultrasound probes can be used for the probe 12A. Therefore, there is no particular limitation on the type of the probe 12A and various types such as convex type, linear type and sector type can be used. The probe may be an external probe or a radial scan type probe for use in an ultrasound endoscope. In addition, the probe 12A may have ultrasound transducers compatible with harmonic imaging for use in receiving second or higher order harmonics from transmitted ultrasonic waves.

The probe 12A includes a piezoelectric unit 16, a signal processor 20, a parallel/serial converter 24, a wireless communication unit 26, an antenna 28, a transmission drive 30, a transmission controller 32, a reception controller 34A, a communication controller 36 and a probe controller 38.

The piezoelectric unit 16 is a one-dimensional or two-dimensional array of (ultrasound) transducers 18 transmitting and receiving ultrasonic waves. The piezoelectric unit 16 is connected to the signal processor 20.

The signal processor 20 includes individual signal processors 20a corresponding to the individual transducers 18 of the piezoelectric unit 16. The individual signal processors 20a are connected to the wireless communication unit 26 via the parallel/serial converter 24. The wireless communication unit 26 is further connected to the antenna 28.

Each of the transducers 18 is connected to the transmission controller 32 via the transmission drive 30. Each of the individual signal processors 20a is connected to the reception controller 34A. The wireless communication unit 26 is connected to the communication controller 36.

The parallel/serial converter 24, the transmission controller 32, the reception controller 34A, and the communication controller 36 are connected to the probe controller 38.

The probe 12A includes a built-in battery, which supplies electric power for drive to each component. The battery is not shown in FIG. 1.

The piezoelectric unit 16 is of a known type which includes a one-dimensional or two-dimensional array of the transducers 18 transmitting and receiving ultrasonic waves, and a backing layer, an acoustic matching layer and an acoustic lens laminated thereon.

Each of the transducers 18 is an ultrasound transducer having a piezoelectric body made of, for example, PZT (lead zirconate titanate) or PVDF (polyvinylidene fluoride), and electrodes provided on both ends of the piezoelectric body.

When a pulsed voltage or a continuous-wave voltage is applied to the electrodes of the ultrasound transducer, the piezoelectric body expands and contracts to cause the transducer to generate pulsed or continuous ultrasonic waves. The ultrasonic waves generated by the ultrasound transducers are combined to form ultrasonic beams.

Upon reception of propagating ultrasonic waves, each ultrasound transducer expands and contracts to produce electric signals, which are then outputted as ultrasonic reception signals.

The transducers 18 transmit ultrasonic waves according to drive signals supplied from the transmission drive 30. The transducers 18 receive ultrasonic echoes from the subject, convert the received ultrasonic echoes into electric signals (reception signals) and output the electric signals to the individual signal processors 20a.

The transmission drive 30 includes a digital/analog converter, a low-pass filter, an amplifier and pulsers. The transmission drive 30 supplies each transducer 18 (electrodes of the ultrasound transducer) with a pulsed drive voltage (transmission pulse) to oscillate the ultrasound transducer to thereby transmit ultrasonic waves.

The transmission drive 30 adjusts the delay amounts of drive signals for the respective transducers 18 based on a transmission delay pattern selected by the transmission controller 32 and supplies the transducers 18 with adjusted drive signals so that the ultrasonic waves transmitted from the transducers 18 form ultrasonic beams.

The transducers 18 of the piezoelectric unit 16 are connected to the corresponding individual signal processors 20a of the signal processor 20.

Each individual signal processor 20a has an AFE (analog front end) including an LNA (low-noise amplifier), a VCA (voltage-controlled attenuator), a PGA (programmable gain amplifier), a low-pass filter and an analog/digital converter. Under the control of the reception controller 34A, the individual signal processors 20a convert the reception signals outputted from the corresponding transducers 18 into digital reception signals in the AFE. Then, the individual signal processors 20a subject the digital reception signals generated in the AFE to quadrature detection or quadrature sampling to generate complex baseband signals. In addition, the individual signal processors 20a sample the generated complex baseband signals to generate sample data containing tissue area information and supply the generated sample data to the parallel/serial converter 24.

The parallel/serial converter 24 converts the parallel sample data generated by the individual signal processors 20a in a plurality of channels into serial sample data.

The ultrasound diagnostic apparatus 10A has the function of spatial compounding in which ultrasound images obtained by the ultrasound transmission and reception (transmission and reception of an ultrasonic wave) in mutually different directions are combined to produce a composite ultrasound image. In the illustrated case, for example, three ultrasound images are combined in spatial compounding. Therefore, when spatial compounding is performed, the reception controller 34A and the transmission controller 32 control the drive of the transmission drive 30 and the individual signal processors 20a, respectively, such that three types of ultrasound transmission and reception are performed in mutually different three directions of transmission and reception.

In cases where a region of interest (hereinafter referred to as “ROI”) is set upon spatial compounding, the reception controller 34A adjusts the depth of the reception signals to be processed in the signal processor 20 in accordance with the set ROI in the ultrasound transmission and reception for obtaining ultrasound images to be combined with a main image which will be described later. This point will be described in detail later.

The wireless communication unit 26 performs carrier modulation based on the serial sample data to generate transmission signals. The wireless communication unit 26 supplies the antenna 28 with the generated transmission signals so that the antenna 28 transmits radio waves to achieve transmission of the serial sample data.

The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 26 uses the antenna 28 to transmit the sample data to the diagnostic apparatus body 14A through wireless communication with the diagnostic apparatus body 14A. The wireless communication unit 26 also receives various control signals (e.g., the ROI to be described later) from the diagnostic apparatus body 14A and outputs the received control signals to the communication controller 36.

The communication controller 36 controls the wireless communication unit 26 so that the sample data is transmitted at a transmission radio field intensity that is set by the probe controller 38. The communication controller 36 outputs various control signals received by the wireless communication unit 26 to the probe controller 38.

The probe controller 38 controls various components of the probe 12A according to various control signals transmitted from the diagnostic apparatus body 14A.

As described above, the ultrasound diagnostic apparatus 10A of the invention has the function of producing an image (composite ultrasound image) through spatial compounding.

As is well known, spatial compounding is a technique which involves performing a plurality of types of ultrasound transmission and reception with respect to a subject in mutually different directions of ultrasound transmission and reception (at mutually different scanning angles or in mutually different scanning directions), and combining ultrasound images obtained by the plurality of types of ultrasound transmission and reception to produce a composite ultrasound image. Such spatial compounding enables speckles of ultrasound images to be reduced.

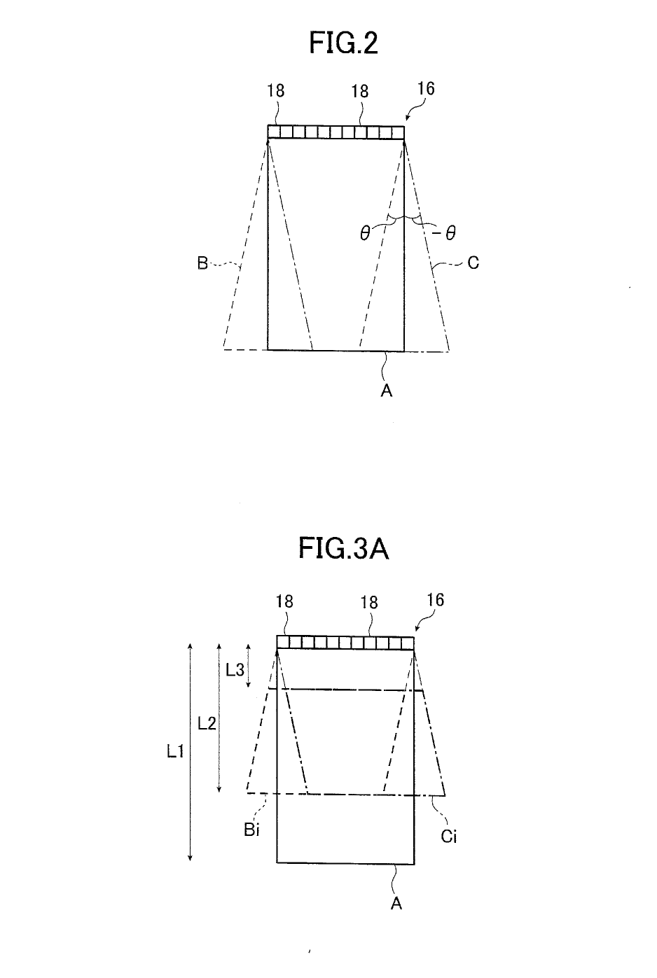

When spatial compounding is performed in the illustrated ultrasound diagnostic apparatus 10A, the probe 12A performs three types of ultrasound transmission and reception in mutually different directions. As conceptually shown in FIG. 2, the three types of ultrasound transmission and reception include, for example, ultrasound transmission and reception for obtaining a main image which is an ultrasound image having the same region as that of a normal ultrasound image (this case is hereinafter referred to as the “transmission and reception for the main image”), ultrasound transmission and reception in a direction inclined by an angle of θ with respect to the direction of the transmission and reception for the main image (ultrasound transmission and reception in the direction inclined by the angle of θ), and ultrasound transmission and reception in a direction inclined by an angle of −θ with respect to the direction of the transmission and reception for the main image (ultrasound transmission and reception in the direction inclined by the angle of −θ).

For convenience, the transmission and reception for the main image is also referred to as the “transmission and reception for an image A”, the ultrasound transmission and reception in the direction inclined by the angle of θ with respect to the direction of the transmission and reception for the image A to as the “transmission and reception for an image B”, and the ultrasound transmission and reception in the direction inclined by the angle of −θ with respect to the direction of the transmission and reception for the image A to as the “transmission and reception for an image C.”

In other words, when spatial compounding is performed in the illustrated example, the three types of ultrasound transmission and reception which make up a frame unit for obtaining a composite ultrasound image are repeatedly performed without changing the frame rate.

Therefore, when spatial compounding is performed, the transmission controller 32 and the reception controller 34A of the probe 12A control the drive of the transmission drive 30 and the individual signal processors 20a, respectively, such that the three types of ultrasound transmission and reception are repeatedly performed.

When spatial compounding is performed, the diagnostic apparatus body 14A (more specifically an image combining unit 80) combines the three ultrasound images including the ultrasound image A (solid line) obtained by the transmission and reception for the image A, the ultrasound image B (broken line) obtained by the transmission and reception for the image B, and the ultrasound image C (chain line) obtained by the transmission and reception for the image C to produce a composite ultrasound image covering the region of the ultrasound image A.

Therefore, in the illustrated example, the number (predetermined number) of ultrasound images to be combined by spatial compounding is 3.

In the practice of the invention, the predetermined number of ultrasound images to be combined by spatial compounding is not limited to 3 but may be 2 or 4 or more.

The method of ultrasound transmission and reception in different directions is not limited to the method as conceptually shown in FIG. 2 in which the ultrasound transmission and reception are delayed. Various known methods of ultrasound transmission and reception in different directions can be used, as exemplified by the methods described in JP 2005-58321 A and JP 2003-70786 A.

In addition, the illustrated example refers to the linear type but, as described above, the invention is applicable to probes of various types including convex type and sector type.

When spatial compounding is performed in the ultrasound diagnostic apparatus 10A of the invention, an arbitrary region in the depth direction (direction of ultrasound transmission and reception) can be set as the ROI where appropriate. In the practice of the invention, a region spaced apart from the piezoelectric unit 16 by a distance which is equal to or larger than a predetermined depth can be set as the ROI.

The ROI is set in, for example, an operating unit 72A of the diagnostic apparatus body 14A to be described later.

In the practice of the invention, the depth from the piezoelectric unit 16 (predetermined depth) which can be set for the ROI is not particularly limited but may be appropriately set according to the characteristics of the piezoelectric unit 16, the main site to be measured, the transmission focal position, the sound field characteristics (near field length) and the like.

The ROI may have a depth that may reach the deeper end of the depth of the main image to be described later if the ROI is spaced apart from the piezoelectric unit 16 by a predetermined depth or more.

In the illustrated example, when the ROI is set in the operating unit 72A, the reception controller 34A of the probe 12A controls the drive of the individual signal processors 20a (the AFEs thereof) for processing the reception signals according to the ROI as for the transmission and reception for the images B and C.

In other words, the ultrasound diagnostic apparatus 10A turns on/off the individual signal processors 20a according to the depth of the ROI to adjust the depth region where the reception signals are to be processed, thereby generating the ROI ultrasound images B and C as ultrasound images to be combined with the ultrasound image A as the main image.

In cases where the ROI is not set upon spatial compounding, the ultrasound diagnostic apparatus 10A generates ultrasound images A to C of the normal depth as shown in FIG. 2 (of the same depth or the same size in the depth direction as the main image) and combines the ultrasound images A to C to produce a composite ultrasound image.

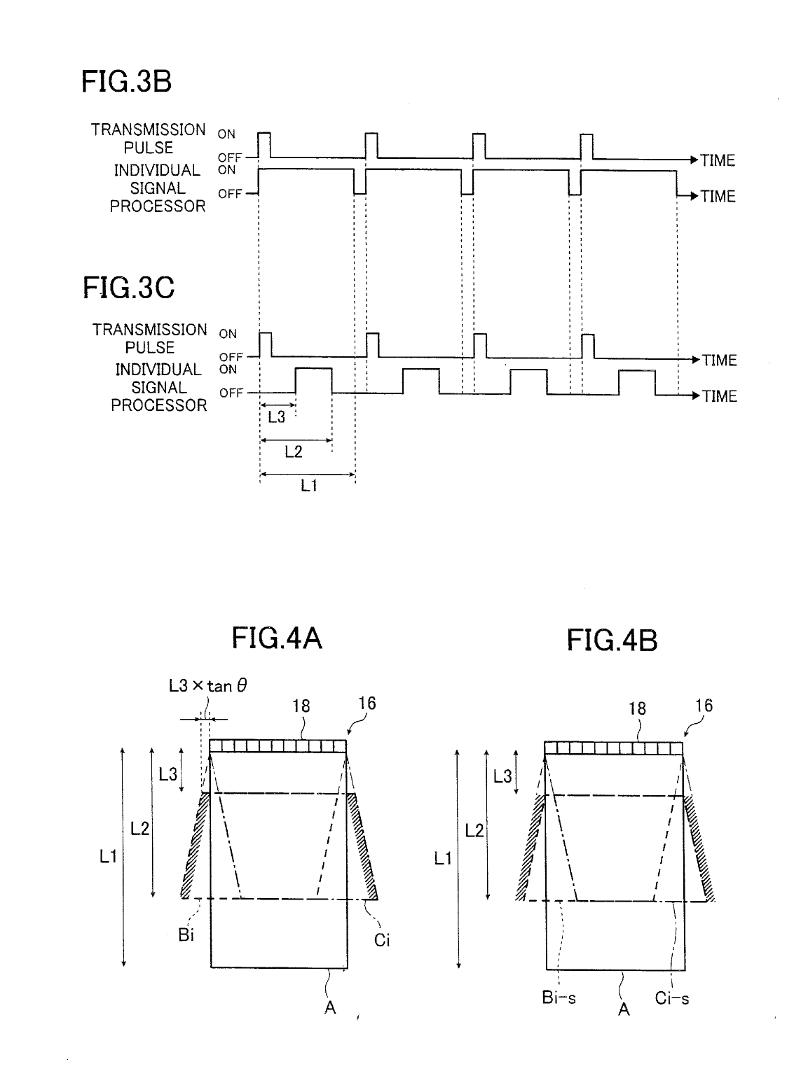

For example, the depth of the transmission and reception for the image A for obtaining the ultrasound image A as the main image is denoted by the depth L1 as conceptually shown in FIG. 3A.

The depth region from the deeper end of the depth L3 to the deeper end of the depth L2 is set as the ROI in the operating unit 72A.

The reception controller 34A of the probe 12A activates or deactivates (on/off) the drive of the individual signal processors 20a of the signal processor 20 according to the depth L1 of the transmission and reception for the image A, and the depths L2 and L3 of the set ROI.

More specifically, in the transmission and reception for the image A, as conceptually shown in FIG. 3B, a transmission pulse is applied while at the same time the drive of the individual signal processors 20a is activated, and the drive of the individual signal processors 20a is deactivated when a time period corresponding to the depth L1 (depth corresponding to the ultrasound image A, that is, the main image) has passed.

On the other hand, in the transmission and reception for the images B and C, as conceptually shown in FIG. 3C, the drive of the individual signal processors 20a is not activated even when a transmission pulse is applied, and the drive of the individual signal processors 20a is activated at a point in time when a time period corresponding to the depth L3 on the shallow side of the ROI has passed. Then, the drive of the individual signal processors 20a is deactivated at a point in time when a time period corresponding to the depth L2 on the deep side of the ROI has passed.

Therefore, in this case, the ultrasound image A as the main image has a rectangular region shown by a solid line in FIG. 3A as in the above example.

In contrast, an ultrasound image Bi in the form of a parallelogram as shown by a thick broken line in FIG. 3A which corresponds to the depth of the ROI is obtained by the transmission and reception for the image B. An ultrasound image Ci in the form of a parallelogram as shown by a thick chain line in FIG. 3A which corresponds to the depth of the ROI is obtained by the transmission and reception for the image C.

As is clear from the above description, the ultrasound diagnostic apparatus 10A of the invention can set an arbitrary ROI in a region spaced apart from the piezoelectric unit 16 by a predetermined depth or more and the image quality of the arbitrarily set ROI can be improved by spatial compounding.

The drive of the individual signal processors 20a for processing the reception signals outputted from the transducers 18 is controlled to generate the ROI ultrasound images to be combined with the main image. Therefore, signal processing can be performed with high efficiency while eliminating the useless reception signal processing. In addition, heat generation from the individual signal processors 20a can also be suppressed as compared to cases where the ultrasound transmission and reception are performed up to the normal depth.

As described above, in cases where the ROI is set upon spatial compounding, the ultrasound diagnostic apparatus 10A processes the reception signals only for the ROI in the ultrasound transmission and reception for at least one image other than the main image, thereby generating an ultrasound image.

As described above, the ROI is a region spaced apart from the piezoelectric unit 16 by a predetermined depth or more. Therefore, as conceptually shown in FIG. 4A, in the ultrasound images Bi and Ci as the ROI images having the depth from the deeper end of the depth L3 to the deeper end of the depth L2, regions occur where the ultrasound images Bi and Ci and the ultrasound image A as the main image do not overlap each other.

In other words, the ultrasound images Bi and Ci corresponding to the ROI and the ultrasound image A as the main image do not overlap each other in the regions corresponding to “L3×tanθ” in terms of the distance in the direction orthogonal to the depth direction as shown by oblique lines in FIG. 4A.

Therefore, it is useless to perform the ultrasound transmission and reception in the regions where the transmission and reception for the images B and C in which the reception signals are processed only for the ROI and those for the image A corresponding to the main image do not overlap each other.

Therefore, in a preferred embodiment of the ultrasound diagnostic apparatus 10A of the invention, as for the ultrasound transmission and reception for obtaining the ROI ultrasound images to be combined with the main image, ultrasound scanning (generation of sound rays) is not performed in the regions where the ultrasound transmission and reception for the main image and those for the ROI ultrasound images do not overlap each other. In other words, ultrasound transmission and reception are not performed in the regions of the ultrasound images to be combined with the main image where the main image and the ultrasound images to be combined with the main image do not overlap each other.

For example in the example shown in FIGS. 4A and 4B, as for the transmission and reception for the images B and C, ultrasound scanning is not performed in the shaded regions shown by the oblique lines to obtain the ultrasound images Bi-s and Ci-s which do not include the shaded regions as shown in FIG. 4B.

When spatial compounding is performed by setting the ROI, the total number of sound rays of the ultrasound images to be combined with the main image can be thus reduced to eliminate the useless ultrasound transmission and reception and efficiently process the reception signals. Heat generation from the individual signal processors 20a can also be suppressed more advantageously.

Instead of ultrasound scanning is not performed, the number of sound rays may be reduced in the regions of the ultrasound images to be combined with the main image where the ultrasound images and the main image do not overlap each other. Alternatively, in the regions of the ultrasound images to be combined with the main image where the ultrasound images and the main image do not overlap each other, the number of available channels may be reduced instead of ultrasound scanning is not performed. Alternatively, in the regions of the ultrasound images to be combined with the main image where the ultrasound images and the main image do not overlap each other, both of the number of sound rays and the number of available channels may be reduced instead of ultrasound scanning is not performed.

In the above examples, when the ROI is set, the images other than the ultrasound image A as the main image are all ROI ultrasound images. However, this is not the sole case of the invention.

In other words, in the practice of the invention, various combinations are possible between the number of ultrasound images having the normal depth and the number of ROI ultrasound images to be combined therewith according to the number (predetermined number) of ultrasound images to be combined by spatial compounding if at least one image is formed according to the set ROI as the ROI ultrasound image.

For example, in the examples shown in FIGS. 2 and 3A-3C, the ultrasound images A and B having the normal depth may be combined with the ROI ultrasound image Ci. Alternatively, the ultrasound images A and C having the normal depth may be combined with the ROI ultrasound image Bi.

The ultrasound image A may also be formed as the image having the ROI depth so that the ultrasound images which are all the ROI ultrasound images are combined together.

In addition, when the ROI is set, only two ultrasound images may be combined together. For example, the ultrasound image A having the normal depth may be combined with the ROI ultrasound image Bi. Alternatively, the ROI ultrasound image Bi may be combined with the ROI ultrasound image Ci.

However, the image for which the ultrasonic waves are transmitted and received in the same directions as in the ultrasound image to be outputted normally is preferably used not as the ROI image but as the image having the normal depth including the predetermined region which serves as the main image to be combined by spatial compounding.

The illustrated probe 12A includes the individual signal processors 20a each of which has the AFE for processing the reception signals (electric signals) outputted from the corresponding transducer 18 which has received the ultrasonic echoes.

As is well known, the integrated circuit such as the AFE processes the signals to generate heat, which may destabilize the operation. As a result, the processing of the reception signals in the individual signal processors 20a is destabilized to deteriorate the quality of the ultrasound images obtained.

Therefore, a temperature sensor (temperature measurement means) may be provided inside the probe 12A so that the number of sound rays and/or the number of available channels (number of transducers 18 to be operated for the ultrasound transmission and reception) can be adjusted according to the temperature measurement results to reduce the quality of the ultrasound images to be combined with the main image.

The temperature sensor is not particularly limited but various known temperature sensors can be used. The temperature sensor preferably measures the temperature of the signal processor 20 having the individual signal processors 20a which are major heat generation sources.

For example, the temperature thresholds including T1 [° C.] and T2 [° C.] which is higher in temperature than T1 are set.

Normal image quality, medium image quality and low image quality are prepared to set the ultrasound image quality. At the normal image quality level, the number of sound rays is 256 and the number of available channels is 64. At the medium image quality level, the number of sound rays is 128 and the number of available channels is 48. At the low image quality level, the number of sound rays is 96 and the number of available channels is 32.

In addition, when the temperature measurement result obtained with the temperature sensor is less than T1, the transmission and reception for the images A, B and C are all performed under the conditions at the normal image quality level.

When the temperature measurement result obtained with the temperature sensor is equal to or more than T1 but less than T2, the transmission and reception for the image A are performed under the conditions at the normal image quality level, whereas those for the images B and C are performed under the conditions at the medium image quality level.

When the temperature measurement result obtained with the temperature sensor is equal to or more than T2, the transmission and reception for the image A are performed under the conditions at the normal image quality level, whereas those of the images B and C are performed under the conditions at the low image quality level.

The temperature increase due to the heat generation within the probe 12A can be thus rapidly suppressed. Heat generation from the probe 12A can also be suppressed heat generation and be suppressed to minimize the deterioration of the image quality. Therefore, this image quality adjusting method enables high-definition ultrasound images to be consistently obtained by spatial compounding.

The conditions of the ultrasound transmission and reception can be adjusted in the same manner according to the temperature of the probe 12A irrespective of whether the ROI is set.

When spatial compounding is performed in the ultrasound diagnostic apparatus 10A of the invention, the order of the ultrasound transmission and reception is not limited to that in which the image A, the image B and the image C are transmitted and received in this order, but the transmission and reception can be performed in various orders.

For example, the ultrasound transmission and reception of the first frame, the second frame, the third frame and the fourth frame and the like may be performed in the orders of “image A→image B→image C”, “image C→image B→image A”, “image A→image B→image C” and “image C→image B→image A” and the like, respectively.

That is, in the practice of the invention, the directions of transmission and reception in the last ultrasound image of one composite ultrasound image in consecutive two frames (i.e., temporally consecutive two composite ultrasound images) and the first ultrasound image of its subsequent composite ultrasound image may be the same. This order of transmission and reception enables the transmission and reception to be continued in the same directions to facilitate the control of the transmission drive 30 and the individual signal processors 20a.

As described above, the reception signals outputted from the probe 12A are supplied to the diagnostic apparatus body 14A by wireless communication.

The diagnostic apparatus body 14A includes an antenna 50, a wireless communication unit 52, a serial/parallel converter 54, a data storage unit 56, an image generating unit 58, a display controller 62, a monitor 64, a communication controller 68, an apparatus body controller 70 and the operating unit 72A.

The antenna 50 for use in the transmission to and reception from the antenna 28 of the probe 12A is connected to the wireless communication unit 52. The wireless communication unit 52 is connected to the data storage unit 56 via the serial/parallel converter 54. The data storage unit 56 is connected to the image generating unit 58. The image generating unit 58 is connected to the monitor 64 via the display controller 62.

The wireless communication unit 52 is connected to the communication controller 68. The serial/parallel converter 54, the image generating unit 58, the display controller 62 and the communication controller 68 are connected to the apparatus body controller 70.

The apparatus body controller 70 controls the components in the diagnostic apparatus body 14A. The apparatus body controller 70 is connected to the operating unit 72A to perform various input operations including as to whether or not spatial compounding is to be performed.

The diagnostic apparatus body 14A includes a built-in power supply unit, which supplies electric power for drive to each component. The power supply unit is not shown in FIG. 1.

The diagnostic apparatus body 14A may include a recharging means for recharging a built-in battery of the probe 12A.

For example, the operating unit 72A of the illustrated ultrasound diagnostic apparatus 10A serves as the means for setting the ROI.

In the ultrasound diagnostic apparatus 10A of the invention, there is no limitation on the method of setting the ROI. Therefore, various known methods of setting and inputting a position and/or a region which are utilized in ultrasound diagnostic apparatuses can be used to set the ROI, as exemplified by a method using a GUI (graphical user interface).

The ultrasound diagnostic apparatus 10A of the invention sets the ROI by inputting the instruction for setting the ROI after the instruction for implementing spatial compounding is issued. Alternatively, spatial compounding for combining ultrasound images may be performed by automatically generating ROI ultrasound images according to the set ROI without the particular need to issue the instruction for implementing spatial compounding.

The wireless communication unit 52 transmits various control signals to the probe 12A through wireless communication with the probe 12A. The wireless communication unit 52 demodulates the signals received by the antenna 50 to output serial sample data.

The communication controller 68 controls the wireless communication unit 52 so that various control signals are transmitted at a transmission radio field intensity that is set by the apparatus body controller 70.

The serial/parallel converter 54 converts the serial sample data outputted from the wireless communication unit 52 into parallel sample data. The data storage unit 56 comprises a memory, a hard disk, or the like and stores at least one frame of sample data converted by the serial/parallel converter 54.

The image generating unit 58 performs reception focusing on sample data for each image read out from the data storage unit 56 to generate image signals representing an ultrasound image. The image generating unit 58 includes a phase adjusting and summing unit 76, an image processor 78 and the image combining unit 80.

The phase adjusting and summing unit 76 selects one reception delay pattern from a plurality of previously stored reception delay patterns according to the reception direction set by the apparatus body controller 70 and, based on the selected reception delay pattern, provides the complex baseband signals represented by the sample data with respective delays and adds them up to perform the reception focusing. This reception focusing yields baseband signals (sound ray signals) where the ultrasonic echoes are well focused.

The image processor 78 generates image signals for an ultrasound image (B-mode image), which is tomographic image information on a tissue inside the subject, according to the sound ray signals generated by the phase adjusting and summing unit 76.

The image processor 78 includes an STC (sensitivity time control) section and a DSC (digital scan converter). The STC section corrects the sound ray signals for the attenuation due to distance according to the depth at which the ultrasonic waves are reflected. The DSC converts the sound ray signals corrected by the STC into image signals compatible with the common scanning method of television signals (raster conversion) and performs required image processing such as gradation processing to generate ultrasound image signals.

The image combining unit 80 combines the ultrasound images generated in the image processor 78 when spatial compounding is performed.

As described above, when spatial compounding is performed, the probe 12A performs the three types of ultrasound transmission and reception for three images, that is, the transmission and reception for the image A, those for the image B and those for the image C.

When spatial compounding is performed, the image combining unit 80 correspondingly combines the ultrasound image A derived from the transmission and reception for the image A, the ultrasound image B derived from the transmission and reception for the image B, and the ultrasound image C derived from the transmission and reception for the image C to generate image signals for a composite ultrasound image.

In cases where the ROI is set upon spatial compounding in the ultrasound diagnostic apparatus 10A of the invention, at least one of the ultrasound images to be combined is an image having the depth of the ROI.

For example, the illustrated example performs spatial compounding of the three images including the ultrasound image A (main image) and the ultrasound images B and C. In cases where the ROI is set upon spatial compounding, as described above, the probe 12A performs the transmission and reception for the image A corresponding to the main image up to the normal depth and changes, according to the ROI, the depth of the reception signal processing of the ultrasonic echoes in the transmission and reception for the images B and C corresponding to the ultrasound images to be combined with the main image. The image combining unit 80 correspondingly combines the ultrasound image A as the main image derived from the transmission and reception for the image A with the ultrasound images Bi and Ci as the images having the depth of the ROI.

The display controller 62 causes the monitor 64 to display an ultrasound image according to the image signals generated by the image generating unit 58.

The monitor 64 includes a display device such as an LCD, for example, and displays an ultrasound image under the control of the display controller 62.

The operation of the ultrasound diagnostic apparatus 10A shown in FIG. 1 is described below.

In the ultrasound diagnostic apparatus 10A, during the diagnosis, various kinds of information inputted from the operating unit 72A of the diagnostic apparatus body 14A are first sent from the wireless communication unit 52 (antenna 50) of the diagnostic apparatus body 14A to the wireless communication unit 26 (antenna 28) of the probe 12A and then supplied to the probe controller 38. Then, ultrasonic waves are transmitted from the transducers 18 in accordance with the drive voltage supplied from the transmission drive 30 of the probe 12A.

The reception signals outputted from the transducers 18 that have received the ultrasonic echoes generated by reflection of the ultrasonic waves on the subject are supplied to the corresponding individual signal processors 20a to generate sample data.

This embodiment refers to the case in which an instruction for spatial compounding is issued using the operating unit 72A and the depth from the deeper end of the depth L3 to the deeper end of the depth L2 as shown in FIG. 3 is set as the ROI.

In the probe 12A, the ROI setting information is sent from the probe controller 38 to the reception controller 34A and the transmission controller 32.

In the probe 12A, upon receipt of such information, the transmission controller 32 controls the drive of the piezoelectric unit 16 (transducers 18) so as to perform the transmission and reception for the images A, B and C. In addition, the reception controller 34A controls the operation of the signal processor 20 (individual signal processors 20a) according to the set ROI so as to process the reception signals for the image A up to the depth L1 as shown in FIG. 3B and to process the reception signals of the images B and C only in the ROI depth from the deeper end of the depth L3 to the deeper end of the depth L2 as shown in FIG. 3C.

Preferably, the transmission controller 32 controls the drive of the transducers 18 and the reception controller 34A controls the operation of the individual signal processors 20a so that the regions of the ROI ultrasound images where the ultrasound images and the main image do not overlap each other are not subjected to ultrasound scanning as shown in FIG. 4B.

The sample data generated by the individual signal processors 20a are sent to the parallel/serial converter 24, where the sample data is converted into serial data. The serial data is then wirelessly transmitted from the wireless communication unit 26 (antenna 28) to the diagnostic apparatus body 14A.

The sample data received by the antenna 50 of the diagnostic apparatus body 14A is sent to the wireless communication unit 52. The sample data is then sent from the wireless communication unit 52 to the serial/parallel converter 54 and is converted into parallel data. The sample data converted into parallel form is stored in the data storage unit 56.

Further, the sample data for each image is read out from the data storage unit 56 to generate image signals of an ultrasound image in the image generating unit 58. The display controller 62 causes the monitor 64 to display the ultrasound image based on the image signals.

When spatial compounding is performed, the image combining unit 80 of the image generating unit 58 combines the ultrasound images.

More specifically, as described above, when spatial compounding is performed, the image combining unit 80 combines the ultrasound image A (main image) derived from the transmission and reception for the image A, the ultrasound image B derived from the transmission and reception for the image B, and the ultrasound image C derived from the transmission and reception for the image C to generate image signals for a composite ultrasound image, and outputs the image signals to the display controller 62.

Since the ROI is set in this embodiment, the image combining unit 80 combines the ultrasound image A as the main image with the ROI ultrasound images Bi (Bi-s) and Ci (Ci-s) to generate image signals of a composite ultrasound image and outputs the image signals to the display controller 62.

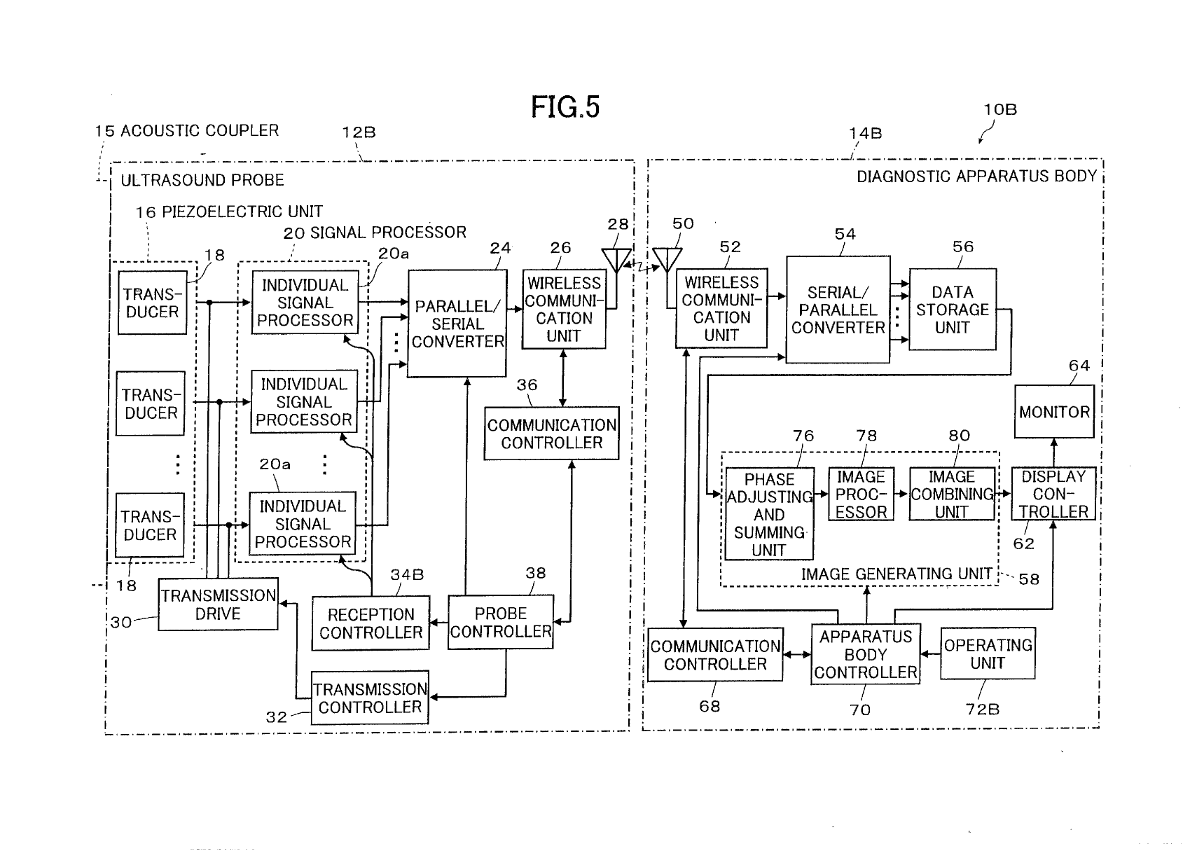

FIG. 5 is a conceptual block diagram showing an embodiment of the ultrasound diagnostic apparatus according to the second aspect of the invention.

Many components of the ultrasound diagnostic apparatus 10B shown in FIG. 5 are the same as those of the ultrasound diagnostic apparatus 10A in the first aspect of the invention shown in FIG. 1. Therefore, like components are denoted by the same reference numerals and the following description mainly focuses on the different features.

As in the above-described ultrasound diagnostic apparatus 10A, the ultrasound diagnostic apparatus 10B shown in FIG. 5 includes an ultrasound probe 12B (hereinafter referred to as “probe 12B”) and a diagnostic apparatus body 14B. As in the above embodiment, the ultrasound probe 12B is connected to the diagnostic apparatus body 14B by wireless communication.

In addition the ultrasound diagnostic apparatus 10B includes an acoustic coupler 15 which is detachably attached to the ultrasound transmission and reception surface of the probe 12B.

The acoustic coupler 15 is used to focus the ultrasonic waves (ultrasonic beams) near the skin surface of the subject. The acoustic coupler 15 is formed of a material having an acoustic impedance close to that of the living body (subject) and is detachably attached to the surface of the probe 12B.

In the practice of the invention, the acoustic coupler 15 is of a known type used in ultrasound diagnostic apparatuses. The acoustic coupler 15 used in the ultrasound diagnostic apparatus 10B of the invention is not limited to one type but a plurality of types of couplers different in thickness and shape may be used for the acoustic coupler 15.

As in the probe 12A, the probe 12B transmits ultrasonic waves to the subject, receives ultrasonic echoes generated by reflection of the ultrasound waves on the subject, and outputs reception signals of an ultrasound image in accordance with the received ultrasonic echoes.

There is no limitation on the type of the probe 12B and various known probes can be used.

As in the probe 12A, the probe 12B also includes a piezoelectric unit 16, a signal processor 20, a parallel/serial converter 24, a wireless communication unit 26, an antenna 28, a transmission drive 30, a transmission controller 32, a reception controller 34B, a communication controller 36 and a probe controller 38.

The probe 12B also includes a built-in battery (not shown), which supplies electric power for drive to each component.

The piezoelectric unit 16, the signal processor 20, the parallel/serial converter 24, the wireless communication unit 26, the antenna 28, the transmission drive 30, the transmission controller 32, the communication controller 36 and the probe controller 38 are basically the same as those of the probe 12A.

More specifically, the piezoelectric unit 16 is a one-dimensional or two-dimensional array of transducers 18 transmitting and receiving ultrasonic waves.

The transmission drive 30 supplies the transducers 18 with a drive voltage so that the transducers transmit ultrasonic waves so as to form ultrasonic beams.

The transducers 18 output the reception signals of the ultrasonic echoes to individual signal processors 20a of the signal processor 20. The individual signal processors 20a process the reception signals to generate sample data and supply the generated sample data to the parallel/serial converter 24. The parallel/serial converter 24 converts the parallel sample data into serial sample data.

The ultrasound diagnostic apparatus 10B also has the function of spatial compounding in which ultrasound images obtained by the ultrasound transmission and reception in mutually different directions are combined to produce a composite ultrasound image.

As in the above embodiment, the ultrasound diagnostic apparatus 10B also combines three ultrasound images when spatial compounding is performed. Therefore, the transmission controller 32 and the reception controller 34B control the drive of the transmission drive 30 and the individual signal processors 20a, respectively, such that three types of ultrasound transmission and reception are performed in mutually different directions of transmission and reception.

In cases where the acoustic coupler 15 is attached to the probe 12B when spatial compounding is to be performed, the reception controller 34B adjusts the depth of the reception signals to be processed in the signal processor 20 in at least one of the ultrasound images to be combined in spatial compounding.

In addition, proximity mode in which images in a predetermined depth region close to the skin surface of the subject are combined by spatial compounding is set in the ultrasound diagnostic apparatus 10B. Also in cases where the proximity mode is instructed, the depth of the reception signals to be processed in the signal processors 20 is adjusted in at least one ultrasound image to be combined by spatial compounding.

This point will be described in detail later.

The wireless communication unit 26 generates transmission signals from the serial sample data and transmits the serial sample data to the diagnostic apparatus body 14B via the antenna 28.

The wireless communication unit 26 receives various control signals (for example regarding the attachment of the acoustic coupler to be described later) from the diagnostic apparatus body 14B and outputs the received control signals to the communication controller 36.

The communication controller 36 controls the wireless communication unit 26. The communication controller 36 outputs various control signals received by the wireless communication unit 26 to the probe controller 38.

The probe controller 38 controls various components of the probe 12B according to various control signals transmitted from the diagnostic apparatus body 14B.

As described above, the ultrasound diagnostic apparatus 10B of the invention has the function of producing an image (composite ultrasound image) through spatial compounding.

As in the ultrasound diagnostic apparatus 10A shown in FIG. 1, the ultrasound diagnostic apparatus 10B also performs, for example, the three types of ultrasound transmission and reception in mutually different directions upon spatial compounding as conceptually shown in FIG. 2. More specifically, when spatial compounding is selected, the probe 12B performs the three types of ultrasound transmission and reception, including the “transmission and reception for the image A” as the transmission and reception for obtaining the main image, the “transmission and reception for the image B” in a direction inclined by an angle of θ with respect to the direction of the transmission and reception for the image A (main image), and the “transmission and reception for the image C” in a direction inclined by an angle of −θ with respect to the direction of the transmission and reception for the image A.

Also in this embodiment, when spatial compounding is performed, the probe 12B repeatedly performs the three types of ultrasound transmission and reception which make up a frame unit without changing the frame rate.

When spatial compounding is performed, the transmission controller 32 and the reception controller 34B of the probe 12B control the drive of the transmission drive 30 and the individual signal processors 20a, respectively, such that the three types of ultrasound transmission and reception are repeatedly performed.

On the other hand, when spatial compounding is performed, the diagnostic apparatus body 14B (more specifically an image combining unit 80) combines the three ultrasound images including the ultrasound image A (solid line) as the main image obtained by the transmission and reception for the image A, the ultrasound image B (broken line) obtained by the transmission and reception for the image B, and the ultrasound image C (chain line) obtained by the transmission and reception for the image C to produce a composite ultrasound image covering the region of the ultrasound image A.

Therefore, the number (predetermined number) of ultrasound images to be combined by spatial compounding in the ultrasound diagnostic apparatus 10B is 3. However, the predetermined number may be 2 or 4 or more as in the above embodiment.

In addition, various known methods can be used to transmit and receive ultrasonic waves in different directions as in the above embodiment.

As described above, the proximity mode in which images in a predetermined depth region near the skin surface of the subject (a predetermined region in the direction of ultrasound transmission and reception) are combined by spatial compounding is set in the ultrasound diagnostic apparatus 10B.

Also in cases where the proximity mode is instructed, the depth of the reception signals to be processed in the signal processors 20 is adjusted in at least one ultrasound image to be combined by spatial compounding.

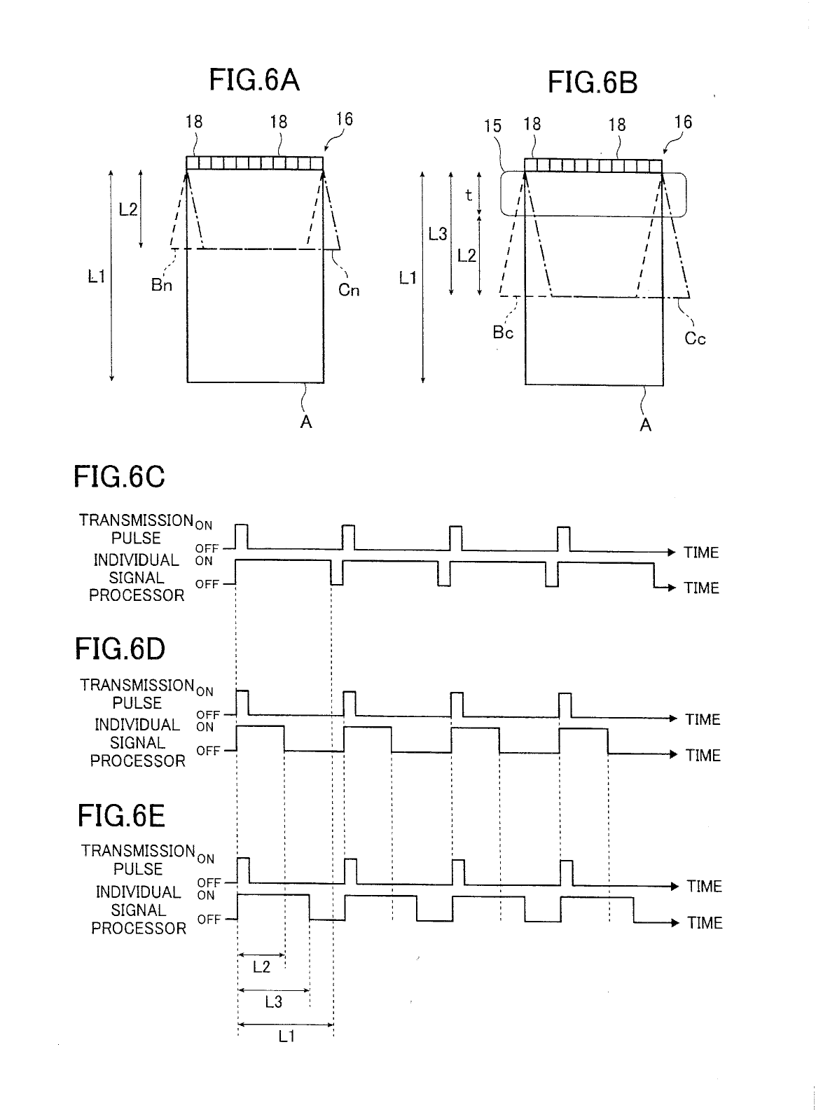

In the illustrated embodiment, the depth L1 (e.g., 5 cm)in normal spatial compounding is set to as conceptually shown in FIG. 6A. Therefore, the ultrasound images A to C are all images having the depth L1 in normal spatial compounding.

In contrast, when spatial compounding is performed in the proximity mode, the transmission and reception for the image A corresponding to the main image are performed up to the depth L1 and those for the images B and C are performed up to the depth L2 (e.g., 2 cm). More specifically, the ultrasound image A having the depth L1 is combined with the ultrasound images Bn and Cn having the depth L2 by spatial compounding in the proximity mode to produce a composite ultrasound image in which the images are combined in the region of the depth L2 near the skin surface of the subject.

In the ultrasound diagnostic apparatus 10B, the drive of the individual signal processors 20a (AFEs thereof) for processing the reception signals is controlled according to the depth of the ultrasound images.

More specifically, in the ultrasound diagnostic apparatus 10B, the reception controller 34B activates or deactivates (on/off) the drive of the individual signal processors 20a of the signal processor 20 according to the depth of the ultrasound images to be combined by spatial compounding to adjust the depth region in which the reception signals are processed, thereby obtaining each ultrasound image as an image having a predetermined depth.

More specifically, in cases where spatial compounding in the proximity mode is selected and instructed by the operation in an operating unit 72B to be described later, as for the transmission and reception for the image A, as conceptually shown in FIG. 6C, a transmission pulse is applied while at the same time the drive of the individual signal processors 20a is activated, and the drive of the individual signal processors 20a is deactivated when a time period corresponding to the depth L1 (depth corresponding to the ultrasound image A, that is, the main image) has passed.

On the other hand, as for the transmission and reception for the images B and C in the proximity mode, as conceptually shown in FIG. 6D, a transmission pulse is applied while at the same time the drive of the individual signal processors 20a is activated, and the drive of the individual signal processors 20a is deactivated at a point in time when a time period corresponding to the depth L2 in the proximity mode which is shorter than the depth L1 has passed.

The ultrasound image A having the depth L1 and the ultrasound images Bn and Cn having the depth L2 can be thus generated.

As described above, the illustrated ultrasound diagnostic apparatus 10B includes the acoustic coupler 15 to focus the ultrasonic waves near the skin surface of the subject.

In the ultrasound diagnostic apparatus 10B, the attachment of the acoustic coupler 15 to the probe 12B is detected by an input operation made with the operating unit 72B of the diagnostic apparatus body 14B to be described later. In other words, in the illustrated embodiment, the operating unit 72B serves as the detector (detection means) for detecting the attachment of the acoustic coupler 15.

Once the attachment of the acoustic coupler 15 is detected in the ultrasound diagnostic apparatus 10B, the probe 12B automatically performs the ultrasound transmission and reception according to the proximity mode, i.e., the generation of ultrasound images according to the proximity mode.

In other words, once the ultrasound diagnostic apparatus 10B detects the attachment of the acoustic coupler 15, the probe 12B automatically transmits and receives ultrasonic waves so that spatial compounding may be only performed near the skin surface of the subject.

Alternatively, in response to the detection of the attachment of the acoustic coupler 15, the probe 12B may simply increase the ultrasound transmission and reception depth of at least one ultrasound image by the thickness of the acoustic coupler 15. Alternatively, in response to the detection of the attachment of the acoustic coupler 15 made when the proximity mode is instructed, the probe 12B may increase the ultrasound transmission and reception depth of at least one ultrasound image by the thickness of the acoustic coupler 15. In addition, these operation modes may be provided so that one of them can be selected.

For example, also in cases where the acoustic coupler 15 is attached, the transmission and reception for the image A (ultrasound image A) is performed up to the depth L1 similarly to the above embodiment as conceptually shown in FIG. 6B.

In contrast, once the acoustic coupler 15 is attached, the transmission and reception for the images B and C is performed up to the depth L3 obtained by adding the depth t (e.g., 1 cm) corresponding to the thickness of the acoustic coupler 15 (the size in the depth direction or in the direction of ultrasound transmission and reception) to the depth L2. In other words, in this case, as shown in FIG. 6B, the ultrasound image A having the depth L1 which is the main image is combined with the ultrasound images Bc and Cc having the depth L3 which is obtained by adding the depth t corresponding to the thickness of the acoustic coupler 15 to the depth L2 in the proximity mode.

Therefore, as in the above embodiment, as for the transmission and reception for the image A, as conceptually shown in FIG. 6C, a transmission pulse is applied while at the same time the drive of the individual signal processors 20a is activated, and the drive of the individual signal processors 20a is deactivated at a point in time when a time period corresponding to the depth L1 has passed.

On the other hand, as for the transmission and reception for the images B and C, as conceptually shown in FIG. 6E, a transmission pulse is applied while at the same time the drive of the individual signal processors 20a is activated, and the drive of the individual signal processors 20a is deactivated at a point in time when a time period corresponding to the depth L3 which is longer by the depth t than the depth L2 has passed.

The ultrasound image A having the depth L1 and the ultrasound images Bc and Cc having the depth L3 can be thus generated.

The case where the acoustic coupler 15 is attached to make an ultrasound diagnosis is namely the case where ultrasound images near the skin surface of the subject are necessary and those in the deep regions are not necessary.

In contrast, according to the invention, once the acoustic coupler 15 is attached, the depth is decreased to a predetermined value or less in at least one of the ultrasound images to be combined by spatial compounding. Therefore, this invention is capable of efficiently obtaining effective high-definition ultrasound images without the need for useless signal processing and generation of sound rays.

In the invention, the operation of spatial compounding near the skin surface of the subject as in the proximity mode is preferably set. In the practice of the invention, it is possible, in the proximity mode, to generate ultrasound images having a depth which is set in consideration of the thickness of the acoustic coupler to thereby produce a composite ultrasound image having a sufficient depth near the skin surface of the subject.

In addition, the drive of the individual signal processors 20a for processing the reception signals outputted from the transducers 18 is controlled to adjust the depth of the ultrasound images. Therefore, useless reception signal processing is eliminated to enable efficient signal processing while controlling useless drive of the AFE and suppressing the heat generation from the individual signal processors 20a.

In spatial compounding with the acoustic coupler 15 attached, the processing of the reception signals is useless to do in the depth region where the acoustic coupler 15 is attached. In other words, in the ultrasound diagnostic apparatus 10B of the invention, it is not necessary to generate ultrasound images by the transmission and reception for the images B ad C in the region of the depth t corresponding to the acoustic coupler 15.

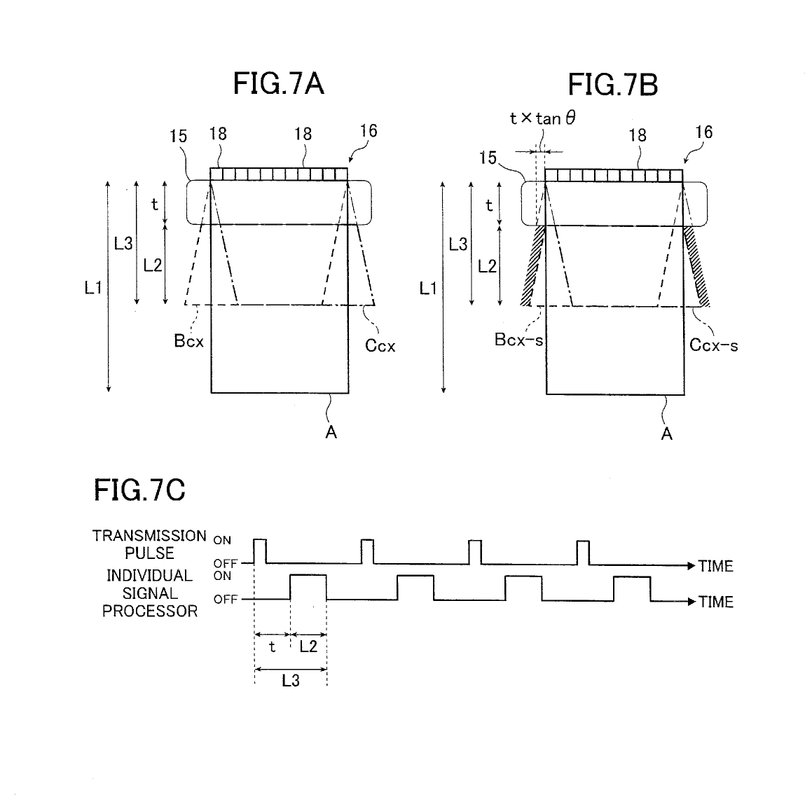

Accordingly, as conceptually shown in FIG. 7A, the ultrasound image A as the main image may be used as the image having the depth L1 and the images obtained by the transmission and reception for the images B and C as the ultrasound images Bcx and Ccx in the region from the deeper end of the depth t to the deeper end of the depth L2 which is obtained by removing the region of the depth t on the probe 12B side from the region of the depth L3.

Therefore, as for the transmission and reception for the image A, the individual signal processors 20a are driven at the timing shown in FIG. 6C as in the above embodiment.

On the other hand, as for the transmission and reception for the images B and C, as shown in FIG. 7C, the drive of the individual signal processors 20a is not activated even when a transmission pulse is applied, and the drive of the individual signal processors 20a is activated at a point in time when a time period corresponding to the depth t which corresponds to the thickness of the acoustic coupler 15 has passed. Then, the drive of the individual signal processors 20a is deactivated at a point in time when a time period corresponding to the depth L3 has passed.

Useless signal processing can be thus further eliminated to perform spatial compounding near the skin surface of the subject with higher efficiency. The heat generation from the signal processor 20 can also be more suppressed.

As shown in FIG. 7B, in the ultrasound images Bcx and Ccx which have no image in the region of the depth t corresponding to the thickness of the acoustic coupler 15, regions occur where these ultrasound images Bcx and Ccx and the ultrasound image A as the image do not overlap each other.

In other words, the ultrasound images Bcx and Ccx and the ultrasound image A as the main image do not overlap each other in the regions corresponding to “t×tanθ” in terms of the distance in the direction orthogonal to the depth direction as shown by oblique lines in FIG. 7B. Therefore, it is no use transmitting and receiving ultrasonic waves in these regions.

Therefore, similarly to the ultrasound diagnostic apparatus 10A, the ultrasound diagnostic apparatus 10B of the invention preferably do not perform the ultrasound transmission and reception in the regions of the ultrasound images to be combined with the main image where the main image and the ultrasound images do not overlap each other. Alternatively, in the regions of the ultrasound images to be combined with the main image where the main image and the ultrasound images do not overlap each other, the number of sound rays and/or the number of available channels may be reduced as in the above embodiment.

For example in the example shown in FIGS. 7A and 7B, as for the transmission and reception for the images B and C, ultrasound scanning is not performed in the shaded regions shown by the oblique lines in FIG. 7B to obtain the ultrasound images Bcx-s and Ccx-s which do not include the shaded region.

When spatial compounding is performed with the acoustic coupler 15 attached, the total number of sound rays of the ultrasound images to be combined with the main image can be thus reduced to eliminate useless ultrasound transmission and reception and efficiently process the reception signals while further suppressing the heat generation from the individual signal processors 20a more advantageously.

The region of the depth t corresponding to the acoustic coupler 15 is useless also in the main image.

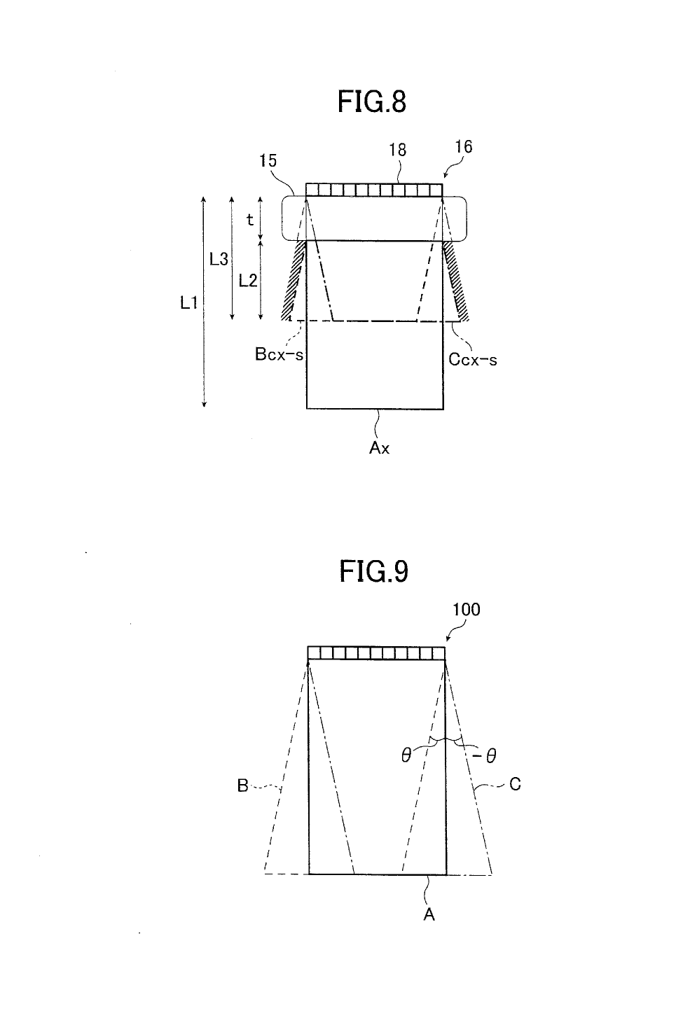

Therefore, in the ultrasound diagnostic apparatus 10B of the invention, the individual signal processors 20a may not process the reception signals in the region of the depth t even in the transmission and reception for the image A as the main image. In other words, an ultrasound image Ax having no image in the region of the depth t corresponding to the acoustic coupler 15 as shown in FIG. 8 may be used as the main image.

This method eliminates more useless signal processing to enable ultrasound images to be efficiently generated by spatial compounding while suppressing the heat generation from the signal processor 20.

The illustrated probe 12B includes the individual signal processors 20a each of which has the AFE for processing the reception signals (electric signals) outputted from the transducer 18. As described above, the integrated circuit including the AFE generates heat by signal processing. The heat generation destabilizes the processing to deteriorate the quality of the ultrasound images obtained.

Therefore, similarly to the probe 12A shown in FIG. 1, the probe 12B may also include a temperature sensor in its interior so that the number of sound rays and/or the number of available channels in the ultrasound transmission and reception can be adjusted according to the temperature measurement results to adjust the quality of the ultrasound images to be combined with the main image to, for example, the above-described normal, medium or low level.

In this way, the temperature increase within the probe 12B can be rapidly suppressed while minimizing the deterioration of the image quality due to the probe 12B.

Also in the ultrasound diagnostic apparatus 10B of the invention, the ultrasonic waves can be transmitted and received in any of various orders when spatial compounding is performed.

In other words, similarly to the ultrasound diagnostic apparatus 10A, also in the ultrasound diagnostic apparatus 10B, the directions of ultrasound transmission and reception in the last ultrasound image in one of consecutive two frames and the first ultrasound image of the subsequent frame may be the same. This order of transmission and reception enables the transmission and reception to be continued in the same directions to facilitate the control of the transmission drive 30 and the individual signal processors 20a.

As described above, the reception signals outputted from the probe 12B are supplied to the diagnostic apparatus body 14B by wireless communication.

Similarly to the diagnostic apparatus body 10A shown in FIG. 1, the diagnostic apparatus body 14B includes an antenna 50, a wireless communication unit 52, a serial/parallel converter 54, a data storage unit 56, an image generating unit 58, a display controller 62, a monitor 64, a communication controller 68, an apparatus body controller 70 and the operating unit 72B.

As in the above embodiment, the diagnostic apparatus body 14B includes a built-in power supply unit (not shown), which supplies electric power for drive to each component.

The antenna 50, the wireless communication unit 52, the serial/parallel converter 54, the data storage unit 56, the image generating unit 58, the display controller 62, the monitor 64, the communication controller 68 and the apparatus body controller 70 are basically the same as those in the diagnostic apparatus body 10A.

More specifically, the wireless communication unit 52 performs wireless communication with the probe 12B via the antenna 50 to transmit control signals to the probe 12B and receive signals sent from the probe 12B. The wireless communication unit 52 demodulates the received signals and outputs them to the serial/parallel converter 54 as serial sample data.

The communication controller 68 controls the wireless communication unit 52 so that various control signals are transmitted according to the settings made by the apparatus body controller 70.

The serial/parallel converter 54 converts the serial sample data into parallel sample data. The data storage unit 56 stores at least one frame of sample data converted by the serial/parallel converter 54.

The image generating unit 58 (phase adjusting and summing unit 76, image processor 78 and image combining unit 80) performs reception focusing on sample data for each image read out from the data storage unit 56 to generate image signals representing an ultrasound diagnostic image.

As described above, when spatial compounding is performed in the ultrasound diagnostic apparatus 10B, the probe 12B performs, for example, the ultrasound transmission and reception for three images, that is, the ultrasound transmission and reception for the images A, B and C.

When spatial compounding is performed, the image combining unit 80 of the image generating unit 58 accordingly combines the ultrasound image A derived from the transmission and reception for the image A, the ultrasound image B derived from the transmission and reception for the image B, and the ultrasound image C derived from the transmission and reception for the image C to generate image signals for a composite ultrasound image.

When spatial compounding is performed in the ultrasound diagnostic apparatus 10B of the invention, once the acoustic coupler 15 is attached to the probe 12B, the depth of at least one of the ultrasound images to be combined is adjusted (is increased).

In the illustrated embodiment, spatial compounding of three images is performed. Alternatively, when the acoustic coupler 15 is attached, the probe 12B performs the transmission and reception for the image A corresponding to the main image to the normal depth L1 and changes the depth of the transmission and reception for the images B and C corresponding to the ultrasound images to be combined with the main image to the depth L3. Alternatively, the ultrasound image Ax having no region of the depth t corresponding to the acoustic coupler 15 may be used as the main image.

The image combining unit 80 accordingly combines the ultrasound image A (Ax) as the main image with the ultrasound images Bc (Bcx, Bcx-s) and Cc (Ccx, Ccx-s) as the ultrasound images near the skin surface of the subject.

The display controller 62 causes the monitor 64 to display an ultrasound image according to the image signals generated by the image generating unit 58.

Under the control of the display controller 62, the monitor 64 displays the ultrasound image.

The apparatus body controller 70 controls the components in the diagnostic apparatus body 14B. The apparatus body controller 70 is connected to the operating unit 72B to perform various input operations including as to whether or not spatial compounding is to be performed.

As described above, a means for informing the probe 12B of the attachment of the acoustic coupler 15 is, for example, set in the operating unit 72B of the ultrasound diagnostic apparatus 10B shown in FIG. 5. In the ultrasound diagnostic apparatus 10B, the attachment of the acoustic coupler 15 is detected through the input operation in the operating unit 72B and the probe 12B is informed of the detection.

There is no limitation on the input method to inform of the attachment of the acoustic coupler 15 in the ultrasound diagnostic apparatus 10B and various methods of inputting information and instructions used in various diagnostic apparatuses can be utilized, as exemplified by a method using a GUI (graphical user interface) and a method which involves providing a dedicated switch to input for and inform of the attachment of the acoustic coupler 15.

In cases where a plurality of types of the acoustic couplers 15 different in thickness, that is, size in the direction of ultrasound transmission and reception are provided, the type of the acoustic coupler 15 used may be inputted so that the thickness, that is, the depth t thereof can be detected. The thickness of the acoustic coupler 15 used may be inputted instead of the type of the acoustic coupler 15.

The method of detecting the attachment of the acoustic coupler 15 is not limited to inputting to the operating unit 72B but various methods can be used.

For example, the probe 12B may be provided with a means for detecting the acoustic coupler 15 so that the attachment of the acoustic coupler 15 to the probe 12B can be detected by this detection means. The detection method is not particularly limited but various known member detection methods can be used, as exemplified by a method using a switch which is turned on or off depending on whether or not the acoustic coupler 15 is attached, a magnetic method, and an optical detection method.

In addition, ultrasonic waves may be used to detect the attachment of the acoustic coupler 15. For example, the ultrasound transmission and reception from and in the transducers 18 are performed and whether or not the acoustic coupler 15 is attached is determined based on the time period from the start of the transmission to the reception of the reflected waves.

The detection means provided in the diagnostic apparatus body 14B such as the operating unit 72B may be used in combination with the detection means provided in the probe 12B.

In the ultrasound diagnostic apparatus 10B of the invention, both of the implementation of spatial compounding and the attachment of the acoustic coupler 15 may be instructed by input operations.

Alternatively, spatial compounding which involves generating the ultrasound image having the depth L3 and combining it with the main image having the depth L1 may be automatically performed at a point in time when the attachment of the acoustic coupler 15 to the probe 12B is detected even if there is no input instruction for spatial compounding.

The operation of the ultrasound diagnostic apparatus 10B shown in FIG. 5 is described below.

Similarly to the ultrasound diagnostic apparatus 10A, during the diagnosis, various kinds of information inputted to the operating unit 72B is first sent to the probe 12B by wireless communication and then supplied to the probe controller 38 also in the ultrasound diagnostic apparatus 10B.

Then, ultrasonic waves are transmitted from the transducers 18 in accordance with the drive voltage supplied from the transmission drive 30 of the probe 12B.

The reception signals outputted from the transducers 18 that have received the ultrasonic echoes generated by reflection of the ultrasonic waves on the subject are supplied to the corresponding individual signal processors 20a to generate sample data.

This embodiment refers to the case in which the acoustic coupler 15 is attached to the probe 12B, an instruction for spatial compounding is issued using the operating unit 72B and an input operation is made to inform of the attachment of the acoustic coupler 15.

Information as to whether spatial compounding is to be performed and information as to whether the acoustic coupler 15 is attached are sent to the probe 12B, and further sent from the probe controller 38 to the reception controller 34B and the transmission controller 32.

Upon receipt of such information, the transmission controller 32 of the probe 12B controls the drive of the piezoelectric unit 16 (transducers 18) so as to perform the transmission and reception for the images A, B and C. In addition, the reception controller 34B controls the operation of the signal processor 20 (individual signal processors 20a) according to the attachment of the acoustic coupler 15 so as to process the reception signals for the image A up to the depth L1 as shown in FIG. 6C and to process the reception signals for the images B and C up to the depth L3 as shown in FIG. 6E. As described above, the reception signals for the image A may be processed from the deeper end of the depth t to the deeper end of the depth L1, and the reception signals for the images B and C may be processed from the deeper end of the depth t to the deeper end of the depth L3.

Preferably, the transmission controller 32 controls the drive of the transducers 18 and the reception controller 34B controls the operation of the individual signal processors 20a so that the regions of the ROI ultrasound images where the ultrasound images and the main image do not overlap each other are not subjected to ultrasound scanning as shown in FIG. 7B.

The sample data generated by the individual signal processors 20a are sent to the parallel/serial converter 24, where the sample data is converted into serial data. The serial data is then wirelessly transmitted from the wireless communication unit 26 (antenna 28) to the diagnostic apparatus body 14B.

The sample data received by the wireless communication unit 52 of the diagnostic apparatus body 14B is converted into parallel data in the serial/parallel converter 54 and stored in the data storage unit 56.

Further, the sample data for each image is read out from the data storage unit 56 to generate image signals of an ultrasound image in the image generating unit 58. The display controller 62 causes the monitor 64 to display the ultrasound image based on the image signals.

When spatial compounding is performed, the image combining unit 80 of the image generating unit 58 combines the ultrasound images.

More specifically, as described above, when spatial compounding is performed, the image combining unit 80 combines the ultrasound image A (main image) derived from the transmission and reception for the image A, the ultrasound image B derived from the transmission and reception for the image B, and the ultrasound image C derived from the transmission and reception for the image C to generate image signals for a composite ultrasound image, and outputs the image signals to the display controller 62.

Since the acoustic coupler 15 is attached in this embodiment, the image combining unit 80 combines the ultrasound image A (Ax) as the main image with the ultrasound images Bc (Bcx, Bcx-s) and Cc (Ccx, Ccx-s) near the skin surface of the subject to generate image signals of a composite ultrasound image and outputs the image signals to the display controller 62.

In the above embodiments, the ultrasound diagnostic apparatus 10A shown in FIG. 1 has the function of spatial compounding according to the set ROI and the ultrasound diagnostic apparatus 10B shown in FIG. 5 has the function of spatial compounding according to the attachment of the acoustic coupler and the function of spatial compounding according to the proximity mode.

However, the ultrasound diagnostic apparatus of the invention is not limited to these configurations. More specifically, the ultrasound diagnostic apparatus of the invention may include the function of spatial compounding according to the set ROI, and the function of spatial compounding according to the attachment of the acoustic coupler. In addition, the ultrasound diagnostic apparatus of the invention may include the function of spatial compounding according to the set ROI, the function of spatial compounding according to the attachment of the acoustic coupler, and the function of spatial compounding according to the proximity mode.

While the ultrasound diagnostic apparatus of the invention has been described above in detail, the invention is by no means limited to the above embodiments, and various improvements and modifications may be made without departing from the scope and spirit of the invention.

What is claimed is:

1. An ultrasound diagnostic apparatus comprising:
an ultrasound probe configured to transmit ultrasonic waves into a subject and receive ultrasonic echoes generated by reflection of the ultrasonic waves from the subject, the ultrasound probe including a signal processor for processing reception signals based on the ultrasonic echoes; and
a diagnostic apparatus body configured to generate ultrasound images in accordance with the reception signals processed in the signal processor of said ultrasound probe and set a region of interest which is spaced apart from said ultrasound probe,
wherein said ultrasound probe is configured to perform a plurality of types of ultrasound transmission and reception in mutually different directions of ultrasound transmission and reception and said diagnostic apparatus body is configured to combine ultrasound images based on each of the plurality of types of ultrasound transmission and reception, and
wherein, upon production of the composite ultrasound image in said diagnostic apparatus body, said ultrasound probe is configured to control drive of said signal processor so that a depth of at least one of said ultrasound images to be combined is changed according to the region of interest.

2. The ultrasound diagnostic apparatus according to claim 1, wherein, upon the production of the composite ultrasound image in said diagnostic apparatus body, said ultrasound probe performs ultrasound transmission and reception for obtaining a main image as an ultrasound image in a preset predetermined output region by one of said plurality of types of ultrasound transmission and reception.

3. The ultrasound diagnostic apparatus according to claim 2, wherein, upon change of a reception depth of at least one image of said ultrasound images to be combined in accordance with the region of interest, said ultrasound probe does not perform ultrasound scanning in a region of said at least one image having the changed reception depth where said at least one image and said main image do not overlap each other.

4. The ultrasound diagnostic apparatus according to claim 2,
wherein said ultrasound diagnostic apparatus comprises a temperature sensor for measuring a temperature at a predetermined position inside said ultrasound probe, and
wherein, upon the production of the composite ultrasound image in said diagnostic apparatus body, said ultrasound probe changes conditions of the ultrasound transmission and reception so as to change an image quality of an ultrasound image to be combined with said main image in accordance with a temperature measurement result obtained with said temperature sensor.

5. The ultrasound diagnostic apparatus according to claim 4, wherein said temperature sensor measures the temperature of said signal processor.

6. The ultrasound diagnostic apparatus according to claim 1, wherein said ultrasound probe transmits and receives ultrasonic waves in identical directions for a last ultrasound image of one composite ultrasound image in temporally consecutive composite ultrasound images and a first ultrasound image of its subsequent composite ultrasound image.

7. An ultrasound diagnostic apparatus comprising:
an ultrasound probe configured to transmit ultrasonic waves into a subject and receive ultrasonic echoes generated by reflection of the ultrasonic waves from the subject, the ultrasound probe including a signal processor for processing reception signals based on the ultrasonic echoes;
a diagnostic apparatus body configured to generate ultrasound images in accordance with the reception signals processed in the signal processor of said ultrasound probe;
an acoustic coupler detachably attached to said ultrasound probe so as to cover an ultrasound transmission and reception surface of said ultrasound probe; and
a detector provided in at least one of said ultrasound probe and said diagnostic apparatus body to detect that said acoustic coupler is attached to said ultrasound probe,
wherein said ultrasound probe is configured to perform a plurality of types of ultrasound transmission and reception in mutually different directions of ultrasound transmission and reception and said diagnostic apparatus body is configured to combine ultrasound images based on each of the plurality of types of ultrasound transmission and reception, and
wherein, upon production of the composite ultrasound image in said diagnostic apparatus body, said ultrasound probe control drive of said signal processor so that a depth of at least one of said ultrasound images to be combined is changed upon detection of attachment of the acoustic coupler to said ultrasound probe made by said detector.

8. The ultrasound diagnostic apparatus according to claim 7, wherein, upon the detection of the attachment of the acoustic coupler to said ultrasound probe made by said detector, said ultrasound probe increases the depth of at least one of said ultrasound images to be combined.

9. The ultrasound diagnostic apparatus according to claim 7, wherein, upon the production of the composite ultrasound image in said diagnostic apparatus body, said ultrasound probe performs ultrasound transmission and reception for obtaining a main image as an ultrasound image in a preset predetermined output region by one of said plurality of types of ultrasound transmission and reception.

10. The ultrasound diagnostic apparatus according to claim 7, wherein, upon the detection of the attachment of the acoustic coupler to said ultrasound probe made by said detector, said ultrasound probe does not process the reception signals in said signal processor as for a depth region corresponding to the acoustic coupler in at least one of said ultrasound images to be combined.

11. The ultrasound diagnostic apparatus according to claim 10, wherein, upon the detection of the attachment of the acoustic coupler to said ultrasound probe made by said detector, said ultrasound probe does not process the reception signals in said signal processor as for the depth region corresponding to said acoustic coupler in all of said ultrasound images for use in producing said composite ultrasound image.

12. The ultrasound diagnostic apparatus according to claim 10, wherein, upon the detection of the attachment of the acoustic coupler to said ultrasound probe made by said detector, said ultrasound probe does not perform ultrasound scanning of regions of other ultrasound images than said main image where the other ultrasound images and the main image do not overlap each other.

13. The ultrasound diagnostic apparatus according to claim 7, which includes a proximity mode for combining said ultrasound images in a predetermined depth region on a subject skin surface side.

14. The ultrasound diagnostic apparatus according to claim 13, wherein, upon the detection of the attachment of the acoustic coupler to said ultrasound probe made by said detector, said ultrasound probe sets the depth of the at least one of said ultrasound images to be combined as a predetermined depth deeper than in said predetermined depth region in said proximity mode.
